(19) United States
(12) Patent Application Publication
(10) Pub. No.: US 2009/0182363 A1
(43) Pub. Date: Jul. 16, 2009

(54) BLOOD VESSEL OCCLUSION AUGER

(75) Inventor: Noam Shamay, Rehovot (IL)

Correspondence Address: FRISHAUF, HOLTZ, GOODMAN & CHICK, PC, 220 Fifth Avenue, 16TH Floor, NEW YORK, NY, 10001-7708, US

(73) Assignee: OVALUM LTD., Rehovot (IL)

(21) Appl. No.: 12/406,753

(22) Filed: Mar. 18, 2009

Related U.S. Application Data
(63) Continuation of application No. PCT/IL2007/001141, filed on Sep. 18, 2007.

(30) Foreign Application Priority Data
Sep. 19, 2006 (IL) 178179

(51) Int. Cl. A61B 17/22

(52) U.S. Cl. 606/159

(57) ABSTRACT

An occlusion auger including a core wire configured for asymmetric deflection when operated to deflect into a deflection state. The deflected core wire has an extrados, a tool nose, and an intrados. When disposed adjacent an occlusion in a vessel, the tool nose and the extrados embed into a respectively, nose depression (ND) and extrados depression (ED) opposite to each other, whereby the vessel is dilated asymmetrically and radially outward for opening a furrow in the occlusion. When released from the deflection state to a released state, the tool nose exits the nose depression and translates distally into the furrow before release of the extrados out of the extrados depression, whereby the tool nose translates distally away from the extrados depression, by one distally step length for each sequence of operation from the deflected state to the released state.

The present application claims the benefit of Israel Patent Application No. 178179 filed on 19 Sep. 2006 and is a Continuation of PCT/IL2007/001141 filed on 18 Sep. 2007

### TECHNICAL FIELD

The present invention relates to devices, systems, and methods for restoring blood flow in occluded blood vessels, and for traversing blood-vessel occlusions.

### DEFINITIONS

Distal refers to both a direction of motion and a location, respectively, a movement in a direction away from an operator or a location at distance from the operator, for example a portion of an instrument located in vivo.

Proximal refers to both a direction of motion and a location, respectively, a movement in a direction toward the operator or a location nearer to the operator, for example a portion of an instrument located ex vivo.

Axial indicates the direction substantially in the longitudinal axis of a blood vessel.

Lateral and radial refer to a direction substantially perpendicular to the longitudinal axis of a blood vessel.

A furrow is considered hereinbelow as being a substantially an axial furrow in a blood vessel.

### BACKGROUND ART

As one out of the many background-art occlusion augers, U.S. Pat. No. 5,741,270 to Hansen et al. discloses a manual actuator for a catheter system for treating a vascular occlusion. Hansen et al. recite a pair of resilient connecting members made of thin strips of resilient material, which regain their original shape after being deformed. Such a configuration operates differently from the present claimed invention, which has no auger tool mechanical elements but includes solely a single resilient longitudinal core wire having a distal extremity operative as an in vivo auger tool for blood vessel dilation and a proximal extremity operative as a force and motion-transmitting element directly operated upon by an auger actuator.

U.S. Pat. No. 4,848,342 to Kaltenbach discloses a rotatable dilation catheter having a pressure member composed of a flexible, torsionally stable element helically wound into a coil having open turns. Such a configuration operates differently from the present claimed invention, which has no auger tool mechanical elements but includes solely a single resilient longitudinal core wire having a distal extremity operative as an in vivo auger tool for blood vessel dilation and a proximal extremity operative as a force and motion-transmitting element directly operated upon by an auger actuator.

The international application No PCT/IL2005/000607 by the same applicant, published as International Publication No. WO 2005/120628, which is incorporated herewith in whole by reference, is considered as being the closest background art. As shown in the background art FIG. 1a, there is disclosed an occlusion auger 1000 having a shaft 130 and a guide wire 120 associated with an auger tool 100 having a plurality of mechanical elements, such as a force applicator 122 and a bow 110, with the occlusion auger being commanded and controlled by an auger actuator 500. Although relatively small, reliable, and easy to operate, the mechanical elements of the auger tool prevent further reduction of size, and preclude enhancement of reliability and improvement of the ease of operation.

It would therefore be advantageous to delete the mechanical elements to achieve a novel auger tool of reduced dimensions, better reliability, and enhanced ease of operation by use of only a single core wire functioning as both the tool auger and the force application element commanded and controlled directly and without intermediary, by the auger actuator.

### DISCLOSURE OF INVENTION

To allow passage into thin blood vessels, it is always an endeavor to implement smaller auger tools. To this end, there is described an auger tool having a distal portion made of a single bent-over thin core wire of some 0.1 mm of diameter for example. Thereby there is provided an auger tool distal portion for in vivo penetration having a largest dimension of about 0.3 mm in the plane of the bent-over core wire and of about the diameter of the core wire, thus 0.1 mm laterally thereto. When the auger tool is deflected within a blood vessel from a first released and straight longitudinal axial state, to a second deflected state, the vessel may be dilated up to some 1.4 mm for example.

The distal portion of the auger tool may also be configured to allow deflection of the distal portion of the core wire out of the two-dimensional plane of the bent-over core wire. This means that the distal portion may deflect into a three-dimensional shape and reach a transverse deflection state to dilate a blood vessel laterally. With a distal portion having a maximal dimension of say 0.3 mm in perpendicular, it may be possible to dilate a blood vessel in orthogonal thereto up to 1.4 mm.

When a symmetric auger tool, or an auger tool for symmetric deflection is introduced into a snuggly fitting blood vessel, it is not possible to achieve an oblique orientation of the auger tool orientation so as to provide for asymmetric vessel dilation, unless auger-tool orientation means are provided to this end. Since it may be desired to achieve asymmetric dilation, the auger tool described hereinbelow is configured for operation in asymmetric deflection.

Furthermore, to minimize dimensions and to increase operational reliability, the single longitudinal core wire is configured as a unitary piece of material having both a distal extremity operative as an in vivo auger tool for blood vessel dilation and a proximal extremity operative as a force and motion-transmitting element directly operated upon by the auger actuator.

### BRIEF DESCRIPTION OF THE DRAWINGS

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements, or parts that appear in more than one figure are preferably labeled with a same or similar number in all the figures in which they appear, in which:

FIG. 1a is a schematic block diagram of a background art occlusion auger,

FIG. 1b is a schematic block diagram of the occlusion auger,

FIG. 2 is a partial side elevation of the occlusion auger, according to FIG. 1,

FIG. 3 is a detail of the side elevation shown in FIG. 2,

FIG. 4a shows an auger tool adjacent an occlusion,

FIG. 4b depicts auger tool in a furrow,

FIG. 5 is a longitudinal cross-section of a blood vessel containing a deflected auger tool,

FIG. 6 is an enlarged detail of the auger tool shown in FIG. 3,

FIG. 7 is an auger tool in navigation mode,

FIG. 8 shows a deflected auger tool,

FIGS. 9 to 13, and 13a detail various embodiments of the auger tool shown in FIG. 3,

FIG. 14 shows a guide wire connector,

FIGS. 15 to 18 inclusive are various embodiments of the guide wire connector,

FIG. 19a depicts a second embodiment of the occlusion auger of FIG. 1,

FIG. 19b depicts a third embodiment of the occlusion auger of FIG. 1,

FIGS. 20 to 23 illustrate details of a second embodiment of the occlusion auger of FIG. 1, and

FIG. 24 shows an example of an additional tool that is guided over the occlusion auger.

FIG. 25a illustrates an auger tool disposed in a vertical plane and configured to allow lateral deflection, and

FIGS. 25b and 25c show an auger tool. respectively, in oblique and in lateral deflection.

### SUMMARY

It is an object of the present invention to provide an auger tool having a single wire, which is operated to sequentially deflect and straighten out to open a crack in a furrow and traverse an occlusion in a blood vessel

It is another object of the present invention to provide a method for implementing an occlusion auger 1000, and an occlusion auger system, operated proximally ex vivo by an auger actuator 500 for distally traversing an occlusion 320 disposed in vivo in a blood vessel 300 having vessel walls 310. There is provided an auger tool 10 with a core distal portion 22 being guided in vivo by a guide wire 30 or equivalent guide 80 or 73, and with a tool nose 27 as the most distal portion of the auger tool, the core distal portion 22 being operable by the auger actuator in an atraumatic repeatable sequence of operation including both deflection to an arcuate state extending radially outward, and release to a released state extending substantially axially straight, and vice versa. There is further provided at least one longitudinally extending unitary core wire 20 having a core distal portion 22, a core proximal portion 22P, a core-returning portion 28, and a core-incoming portion 24. The core wire has a core distal extremity 29 coupled in continuation to a distal portion of the guide wire or equivalent guide for causing asymmetric deflection of the core distal portion when operated to deflect to the arcuate state. The core proximal portion is coupled directly to the auger actuator 500. Operating the auger actuator for urging the core distal portion into the arcuate state causes the core-returning portion to deform into a longitudinal extrados 34) and the core-incoming portion to deform into a longitudinal intrados 35. Disposing the core distal portion axially in the blood vessel and adjacent the occlusion causes the tool nose and the extrados to embed and become releasably retained in, respectively, a nose depression ND and an extrados depression ED which are disposed in opposite to each other in spaced-apart relationship in the blood vessel. Thereby the vessel is dilated asymmetrically in radial outward direction for opening a furrow 340 in the occlusion.

### BEST MODE FOR CARRYING OUT THE INVENTION

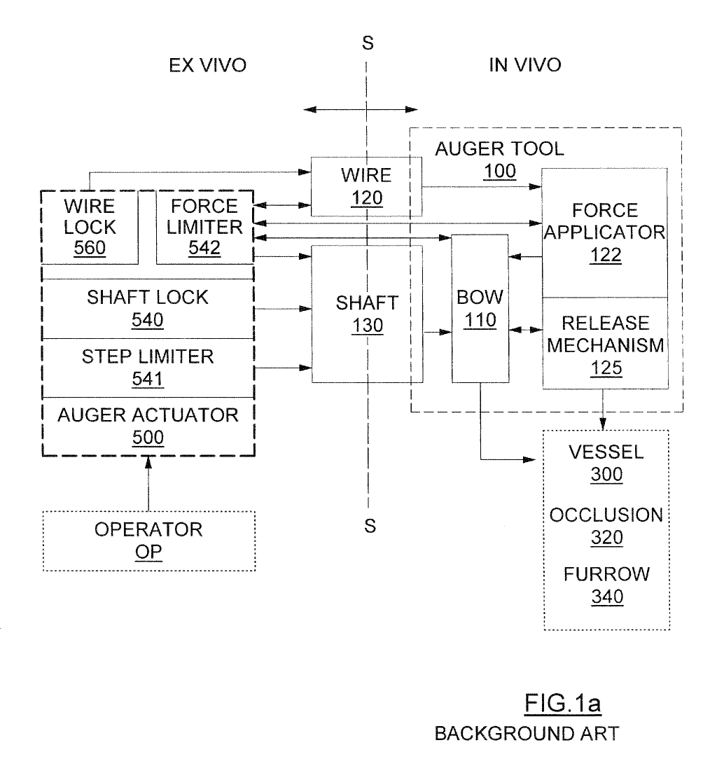

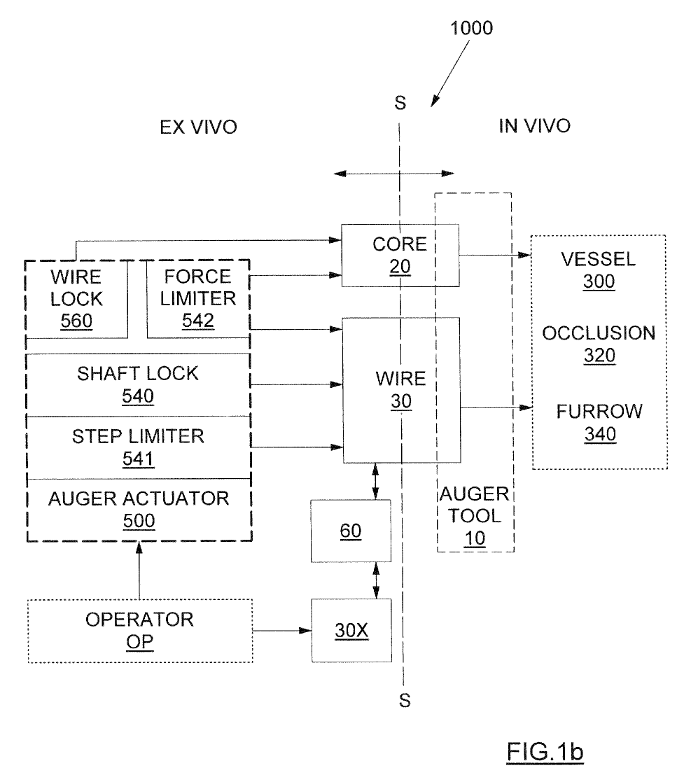

FIG. 1b presents a block diagram showing the mutual relationship and the main elements of an occlusion auger 1000, or occlusion auger system 1000 when in operation, with a proximal ex vivo portion, and a distal in vivo portion, shown separated by a dashed line S-S. A guide wire 30, or wire 30, and a core wire 20, or core 20, both have a proximal ex vivo portion and a distal in vivo portion. A guiding catheter wherethrough the occlusion auger 1000 is introduced into the patient, and the patient, are not shown in FIG. 1b.

The auger actuator 500 shown schematically in FIG. 1b, is operated by an operator OP but is not described in detail since being similar to the auger actuator 500 described in the incorporated international application publication No. WO 2005/120628. The auger actuator includes a main resilient element, and a navigation mode setting, both not shown in the Figs.

The operator OP is able to operate the wire 20 and the core 30 to guide and control an in vivo auger tool 10 inserted in to a blood vessel 300. When adjacent an occlusion 320, the auger tool is operated to open a furrow 340 and traverse the occlusion 320.

If desired, an extension wire 30X may be coupled in co-alignment to the wire 30 by help of a wire connector 60, as described hereinbelow.

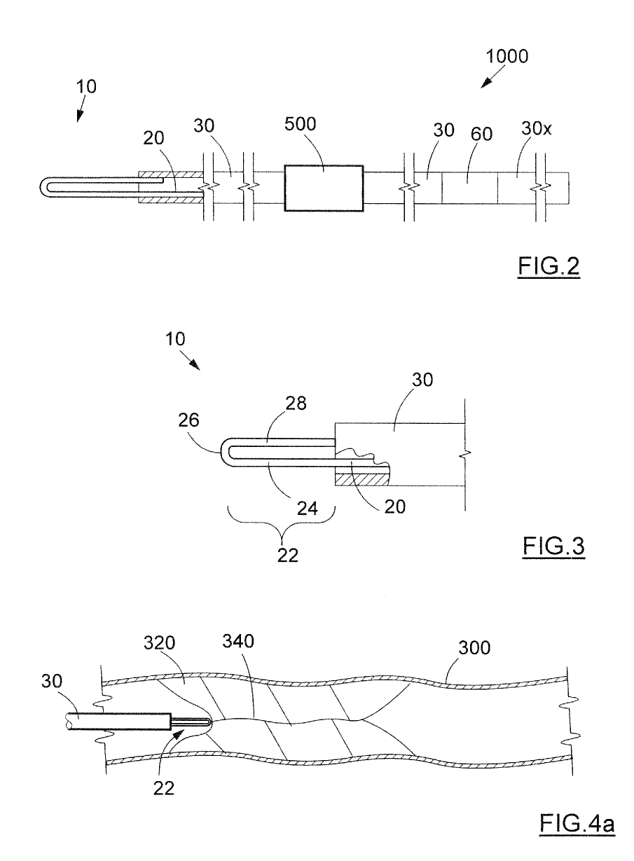

FIG. 2 illustrates the occlusion auger 1000 having a distal auger tool 10, a guide wire 30 forming passage for the core wire 20, an auger actuator 500, a wire connector 60, and an extension wire 30X.

An operator OP, not shown in the Figs., may handle the occlusion auger 1000 via the ex vivo auger actuator 500, which controls the in vivo auger tool 10 in association with a distal portion of the core wire 20 and of the guide wire 30. The at least one resilient core wire 20 is thus configured as a unitary piece of material having both a distal extremity operative as an in vivo auger tool 10 for blood vessel dilation and a proximal extremity operative as a force and motion transmitting element directly operated upon by the auger actuator 500. This unitary single piece of material configuration of the core wire 20 is advantageous for minimizing auger tool dimensions and for increasing operational reliability.

In FIG. 3, in a first exemplary embodiment, the in vivo auger tool 10 may have only a core distal portion 22, which includes the core wire 20 bent like a hairpin that extends distally away out of the guide wire 30. The core wire 20 is an extremely thin, flexible, and resilient element, and the core distal portion 22 may include a core-incoming portion 24, a bend 26, and a core-returning portion 28. For example, the core wire 20 may have a diameter of 0.1 mm. and the maximum dimension of the core distal portion 22 in the plane defined thereby, may reach 0.3 mm.

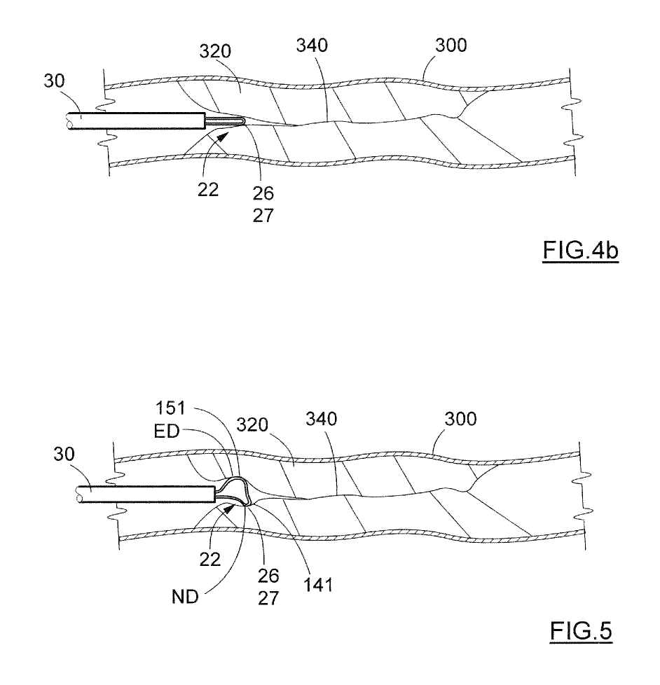

In operation, the core 20 may first be navigated distally via blood vessels 300, inside vessel walls 310, until the bend 26 engages an occlusion 320, as shown in FIG. 4a, and penetrate into a furrow 340 in an occlusion 320, as depicted in FIG. 4b.

Then, the auger tool 10 may be operated to flex asymmetrically and arcuate the core returning portion 28, as shown in FIG. 5. Thereby, the arcuate flexed core-incoming portion 24 forms a longitudinal and continuous linear intrados 35 that may push the bend 26, or tool nose 27 to embed in a nose depression ND, in releasable anchoring disposed in, or adjacent, the occlusion tissue lining the furrow 340. Thereafter, the arcuate flexed and continuous core-returning portion 28 may become embedded in an extrados depression ED, proximal to the nose depression ND, in an opposite vessel wall 310 in or near the occlusion tissue lining the furrow 340.

When operated, flexing the core-returning portion 28 dilates the furrow 340 asymmetrically into one radial direction, providing forces to provoke and initiate a crack propagation mechanism in the occlusion 320, and to further open and distally deepen the furrow 340. Next, forces on the core-returning portion 28 are released whereby the elastic energy accumulated therein while flexing and the elastic energy accumulated in the main resilient element disposed in the auger actuator 500 are liberated, allowing to expand the core-returning portion 28.

When the auger tool 10 is expanded and release straight, the embedded longitudinal extrados 34 of the arcuate core-returning portion 28 may provide support for the bend 26 which may be released first to become introduced by a one step-length distally deeper into the furrow 340. Thereafter, the arcuate core-returning portion 28 is straightened to the released state.

The flexure to arcuate, and the release to expand the core-returning portion 28 are respectively, a first and a second state of a sequence having two phases, controllably operated in successive repetition by the auger actuator 500 to cross the occlusion 320. If necessary, repetitive operation of a plurality of sequences is reiterated successively to traverse one or more occlusions. Figuratively, the auger tool 10 coils-up when flexing, and uncoils when expanding, to progresses distally in a worm-like type of crawling process.

There is thus provided a method for implementing an occlusion auger 1000 and an occlusion auger system 1000 which are operated, commanded and controlled proximally ex vivo by an operator OP handling an auger actuator 500 for distally traversing an occlusion 320 disposed in vivo in a blood vessel 300 having vessel walls 310. The method and the system comprise providing an auger tool 10 with a core distal portion 22 to be guided in vivo by a guide wire 30 or equivalent guide, with a tool nose 27 being the most distal portion of the auger tool. The core distal portion 22 is operable by the auger actuator in an atraumatic repeatable sequence of operation including both deflection to an arcuate state extending radially outward, and release to a released state extending substantially axially straight, and vice versa.

The method and the system comprise the following steps. First, configuring at least one longitudinally extending core wire 20 as a unitary wire having a core distal portion 22, a core proximal portion 22P, a core-returning portion 28 and a core-incoming portion 24. The core wire includes a core wire extremity 29 coupled in continuation to a distal portion of the guide wire or equivalent guide for causing asymmetric dilation of the core distal portion when operated to deflect to the arcuate state. Furthermore, the core proximal portion of the core wire is coupled directly to the auger actuator 500.

Second, the auger actuator is operated for urging the core distal portion into the arcuate state, which causes the core-returning portion to deform in a longitudinal continuous extrados 34 and the core-incoming portion to deform into a longitudinal continuous intrados 35.

Therefore, when disposing the core distal portion axially in the blood vessel and adjacent the occlusion, this causes the tool nose and the extrados to embed and become releasably retained in, respectively, a nose depression ND and an extrados depression ED which are disposed in opposite to each other in spaced-apart relationship in the blood vessel, whereby the blood vessel is dilated asymmetrically in radial outward direction for opening a furrow 340 in the occlusion.

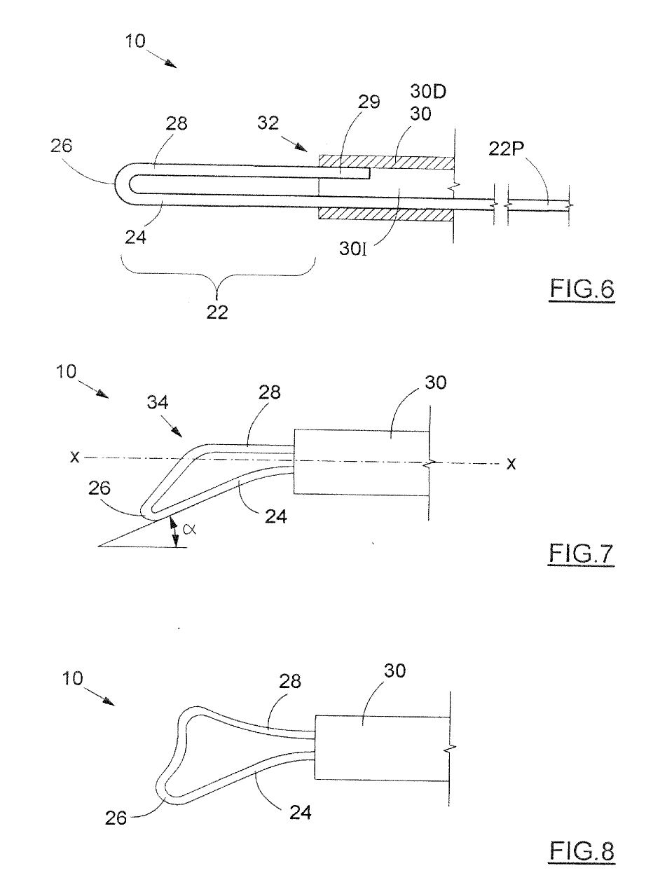

FIG. 6 shows a side elevation and partial cross-section view of the in vivo portion of the auger tool 10, as one embodiment of a portion of the occlusion auger 1000, according to FIG. 1. The auger tool 10 may include a guide wire 30, such as that of an available intravascular guidewire system for the treatment of occluded blood vessels. The core 20 may be introduced through a first proximal wire opening entered in the wire 30 but not shown in FIG. 6, and may exit out of a second distal wire opening 32 as a core distal portion 22.

The core 20 that exits out of the interior 30I of the wire 30 is the distal core-incoming portion 24, which is terminated by a semicircular arc to form the bend 26. The portion of the core 20 extending from the bend 26 and returning back into the distal wire opening 32 is the distal core-returning portion 28, which is terminated by the core distal extremity 29. At the distal guide wire opening 32, the core distal extremity 29 is firmly coupled into the interior 30I of the wire 30. The core distal portion 22 thus looks like a hairpin with a distal bend 26 and proximal thereto, two legs, which are the core-incoming portions 24 and the core returning portion 28.

The core distal extremity 29 of the core-returning portion 28 is firmly and fixedly secured to the guide wire interior 30I adjacent to the distal guide wire opening 32. This firm retention is achieved by any of the means known in the art, such as brazing, welding, laser welding, gluing, or any other method. If desired, the core distal extremity 29 is treated accordingly, such as appropriately flattened, or curved to facilitate firm retention to the wire 30.

The core 20 thus extends through the wire 30, starting at least from the auger actuator 500 and out of the distal wire opening 32 where the core distal portion 22 forms the auger tool 10. Since the core distal extremity 29 is firmly secured to the guide wire interior 30I adjacent to the distal wire opening 32, the core distal portion 22 will rotate together with the wire 30 when this last one is rotated.

At rest, in the released extended and straightened-out state, the core distal portion 22 is disposed in coextensive alignment with the wire 30. To flex the core-returning portion 28 into an arcuate state extending radially away from the wire 30, it is necessary to operate the auger actuator 500 to induce buckling of the core-returning portion 28. Force for buckling the core-returning portion 28 asymmetrically, and also for curving the core incoming portion 24, is supplied via the auger actuator 500, which is operated to decrease the distance between the core distal extremity 29 and the bend 26 and thereby force the core-returning portion 28 to flex.

Preferably, the auger actuator 500 is handled to retain the core 20 locked and push the wire 30 distally away so that the core-returning portion 28 will buckle and deflect between the distal wire opening 32 and the bend 26. The same result is achieved by using the auger actuator 500 to lock the wire 30 and pull the core-incoming portions 24 proximally in.

When the auger actuator 500 is handled to push the wire 30 distally away, energy is accumulated at least in the core-returning portion 28 and in a main resilient actuator element 502, not shown in the Figs., such as a coil spring disposed in the interior of the actuator 500. Therefore, once the actuator 500 is commanded by the operator OP to release the force applied to buckle the core-returning portion 28, energy released from both the core-returning portion 28 and the main resilient actuator element 502 will return the core distal portion 22 to its extended and elongated state in co-alignment with the wire 30.

In operation, when deflected into in the arcuate state, the tool nose 27 embeds in a nose depression ND and the extrados 34 embeds in an extrados depression ED, and when released to the released state, the core distal portion 22 releases the tool nose out of the nose depression ND and causes distal translation thereof into the furrow before release of the extrados out of the extrados depression. Thereby the tool nose translates into the furrow distally away from the extrados depression, by one distal step length for each one sequence of operation.

Each next sequence of operation of the auger tool is accompanied by a next distal nose depression, and by a next distal extrados depression, and both the next distal nose depression and the next extrados depression are disposed distally away relative to, respectively, a previous nose depression and a previous extrados depression.

In other words, the longitudinal core-returning portion 28 is configured for flexing into an asymmetric controlled deflection curve, and operation of the auger actuator induces an atraumatic crawling motion including radial outward dilation, and distal translation. This means that the operation of the auger tool in a specific number of successive sequences is accompanied by a same specific number of radial outward dilations and of distal translations. Thereby, the auger tool translates substantially axially and distally into the blood vessel in successive crawling motion imparted by each successive sequence of operation of the auger actuator.

Therefore, in the arcuate state, the core distal portion is releasably embedded in a nose depression, and the extrados is releasably embedded in an extrados depression to dilate the furrow, and to initiate a crack propagation mechanism to open and distally deepen the furrow. Then, in the released state, the tool nose is received by a one step length distal translation deeper into the deepened furrow.

It is noted that the nose depression is disposed opposite the extrados depression, and that the extrados depression may span a length disposed differently relative to the nose depression. The disposition of the extrados depression relative to the nose depression may be selected from the group consisting of a length extending from proximally to distally relative to the nose depression, a length extending proximally relative to the nose depression, and a length extending distally relative to the nose depression.

In addition to a first control position dedicated to the normally extended state shown in FIG. 6, and to a second control position reserved for the flexed state of the core-returning portion 28, the auger actuator 500 also has an intermediate control position permitting to dispose the auger tool 10 in a navigation state.

FIG. 7 depicts the navigation mode of the auger tool 10 showing the partially arcuate longitudinal asymmetric extrados 34 of the core-returning portion 28 when in a partially asymmetric deflected state.

In FIG. 7 the core distal portion 22 is thus seen flexed asymmetrically in the navigation mode, which is a partial deflection intermediate the fully extended and straightened state and the fully arcuate state where the core-returning portion 28 is completely deflected. In the navigation mode the core distal portion 22 is deflected radially away relative to the longitudinal axis X-X of the distal portion of the wire 30 and forms therewith an angle indicated as a in FIG. 7. Thereby, when the wire 30 is rotated, the core 20 rotates therewith, and the core-incoming portion 24 becomes a directrix that describes the mantle of a cone permitting to orient the bend 26 in any desired direction, thus allowing to properly aligning the auger tool 10 to easily penetrate into a sideways branching blood vessel. Therefore, when the auger tool 10 is disposed in the interior of a vessel 300, and the operator OP desires to introduce the core distal portion 22 past a curve or into a branch of a vessel 300, the wire 30 is rotated until the bend 26 is suitably oriented to point into the direction appropriate to proceed past a curve or into a branch.

FIG. 8 shows the auger tool 10 in the deflected mode with the core distal portion 22 in the asymmetric fully deflected state wherein the arcuate longitudinal extrados 34 of the core-returning portion 28 is clearly seen.

To ensure proper operation of the auger tool 10, it is preferable to delimit the flexure of the core-returning portion 28 between the bend 26 and the core distal extremity 29. The dispositions taken for coupling the core distal extremity 29 to the wire 30, such as a flattening of the core 20 are usually sufficient to serve as a incontinuous point connection where flexing will start. Eventually, a rigidity-weakening feature or a bend-inducing plastic deformation may be entered to the core distal extremity 29 to initiate flexing.

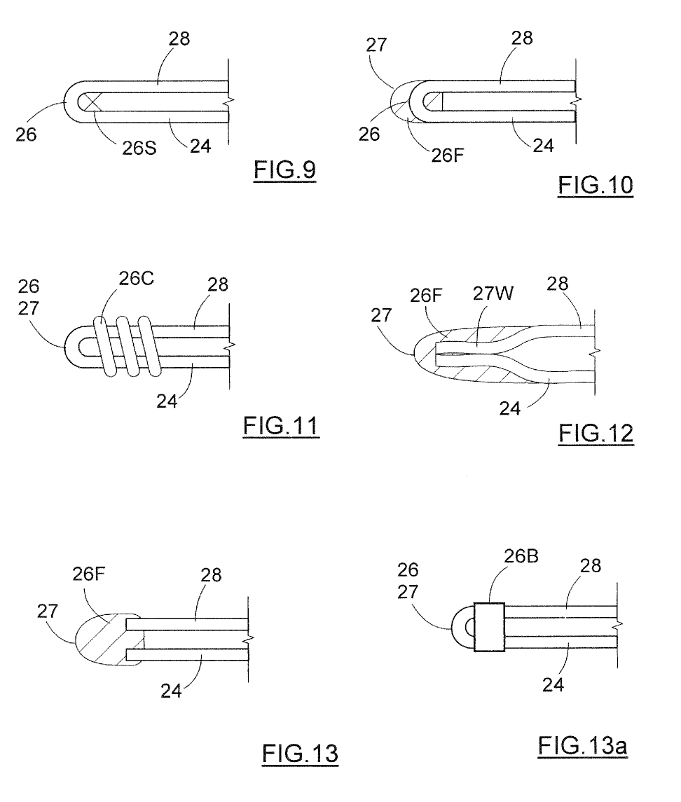

The bend 26 may be stiffened so that the core-returning portion 28, now weaker and less rigid relatively to the stiffened bend portion will deflect adjacent thereto. For example, the bend 26 may be stiffened as indicated by the stiffened bend 26S. say by welding, or gluing, or otherwise stiffened, as shown symbolically in FIG. 9.

FIG. 10 presents another manner to stiffen the bend 26 by providing a fairing 26F as a cover cap for enhancing translational penetration capability of the auger tool 10 in a furrow 340 and through an occlusion 320. The fairing cover 26F is possibly implemented by application of a potting material, or of a glue G, or of a UV cured material, or of any suitable cap appropriately coupled to the bend 26. When the bend 26 is covered by a fairing 26F the most distal portion of the auger tool 10 is indicated as the tool nose 27. According to the selected embodiment, the tool nose 27 indicates the most distal portion of the auger tool 10, which is either the bend 26, in absence of a fairing 26F, or the distal tip of the fairing 26F.

FIG. 11 illustrates a wrapped coil 26C coupling the core-incoming portions 24 and the core returning portion 28 and disposed adjacent the bend 26. In addition to the stiffening action, the wrapped coil 26C also prevents stress concentration at the bend 26. Although not shown in the Figs., other means known to the art are applicable to stiffen, cover, and relieve stress at the bend 26. For example, although not shown in the Figs., a fairing 26F may possibly be combined with a wrapped coil 26C.

FIGS. 12, 13, and 13a present further embodiments of details of core distal portion 22 configurations.

In FIG. 12 the bend 26 is replaced by a coupling, say by welding, or by other means, that firmly connects a short length of core-returning portion 28 that is welded to the core-incoming portions 24 and is covered by a fairing 26F. Instead of the bend 26 there is now a weld 27W where the distal portion of the core-incoming portions 24 is coupled to a short length of core wire, which is now the core-returning portion 28. The weld 27W is covered by the fairing 26F for which the distal extremity is the tool nose 27. Proximally to the fairing 26F both the core-incoming portions 24 and the core-returning portion 28 may be parallel or not to each other and operate as described hereinabove.

FIG. 13 is a further embodiment of a core distal portion 22 configuration shown with the core-incoming portions 24 and a length of core returning portion 28 being firmly spaced apart and coupled together by the fairing 26F. If desired, a wrapped coil 26C is added proximally to the fairing 26F.

FIG. 13a is yet a further embodiment of a core distal portion 22 configuration where the wrapped coil 26C shown in FIG. 11 is replaced by a flat surrounding metal band 26B. The surrounding band 26B operates like the wrapped coil 26C and although not shown in the Figs., other means known to the art are applicable to stiffen, cover, and relieve stress at the bend 26. For example, a fairing 26F is possibly combined with the surrounding band 26B. The incoming portion 24 and the returning portion 28 may thus be coupled by a connection selected alone and in combination at least from the group consisting of a bend 26, of a surrounding band 26B, of a wrapped coil 26C, of a fairing cover 26F, and of a weld 26W. Other modes of connection may also be found to be practical.

To conclude, the core-returning portion may be configured for direct operative association with the guide wire, to first flex the core returning portion to the arcuate state when the guide wire is translated distally relative to the auger actuator, thereby causing the extrados to dilate the vessel in asymmetric radial outward direction, and second, to release the core returning portion to the released state when the guide wire is released. Thereby the tool nose is caused to translate distally away relative to the extrados depression by one predetermined step length for each one sequence of operation.

Described differently, the core distal portion may be navigated in a first step, to engage an axial furrow of an occlusion disposed in a blood vessel. In a second step, the auger actuator may be operated to deflect the core distal portion to the arcuate state, whereby the deflected core returning portion dilates the vessel asymmetrically and radially outward. As a third step, the auger actuator may be operated to return the core distal portion to the released state, whereby the core distal portion translates the tool nose distally into the furrow. Finally, the sequence of the second and third step may be repeated successively until the occlusion is traversed.

The core 20 is possibly made of a super-flexible and super-elastic resilient material such as Nitinol for example. It is noted that the fairing 26F, the surrounding band 26B, and the wrapped coil 26C are possibly implemented from or may include a radio opaque material so that an operator OP viewing an imaging system, for example a Computer Tomography (CT) or a Radiograph, may visualize the disposition of the opaque material with respect to say an occlusion 320.

The auger actuator 500 disposed ex vivo in operative association with the auger tool 10 described in detail in the application incorporated herewith in whole, includes inherent force limiting mechanisms configured for the adjustable selection and for the setting of a predetermined threshold limit of forces applied to the core wire 20 and to the guide wire 30. There is also a step limiter configured for the adjustable selection and for setting of a predetermined distal step length identically taken and repeated in each one sequence of operation. It is not the operator OP but the auger control, or auger actuator 500 that maintains identical predetermined force limits and step-length settings for each sequence in a series of successively repeated sequences.

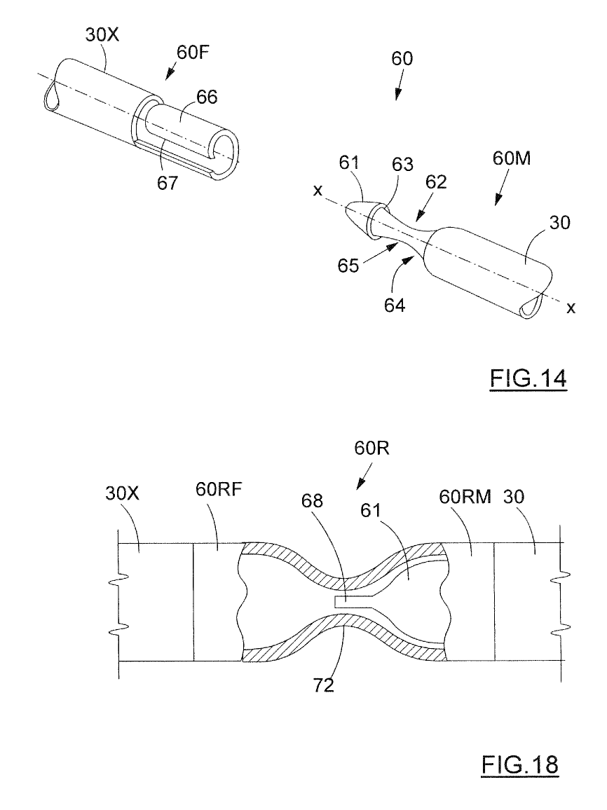

FIG. 14 shows an embodiment of the guide wire connector 60 configured for the fast and firm releasable connection of an additional length of extension wire 30X to the guide wire 30. Both the guide wire 30 and the extension wire 30X have the same outer diameter and may be used as a rail for guiding thereover of one or more devices, such as catheters, tool carrying devices, and operation and treatment tools in general.

The guide wire connector 60 has a male element 60M and a female element 60F configured for quick assembly providing secure but releasable retention by axial introduction of the male element 60M into the female element 60F. The guide wire connector 60 uses a radially curved leaf spring loaded axial snap-in locking mechanism providing disconnection by bending either one of both the male element 60M and the female element 60F relative to each other, whereby snap-out is achieved. In the example described hereinbelow, although the male element 60M is shown coupled to the guide wire 30 whereas the female element 60F is disposed on the extension wire 30X, the situation may be reversed, whereby the male element 60M is attached to the extension wire 30X and the female element 60F is coupled to the guide wire 30.

In FIG. 14 the axisymmetric male element 60M is shown with a front ogive 61, having an outer diameter smaller than that of the wire 30, and disposed in coaxial alignment with the wire 30. However, if desired instead of an ogive 61, a conical shape is also practical. A groove 62 forming shoulders perpendicular to the axis X-X of the male element 60M is cut between the ogive 61 and the wire 30, thereby creating a first shoulder 63 adjacent the ogive 61 and a second shoulder 64 adjacent the wire 30. The axial distance between the first shoulder 63 and the second shoulder 64 defines the length of the groove 62.

The depth of the groove 62 is selected to match the requirements of the female element 60F, as will be described hereinbelow. The bottom 65 of the groove 62 preferably presents a varying radius, so that the diameter of the groove is larger at the shoulders and smaller between them. In other words, the diameter 65 of the bottom of the groove 62 is smaller in the middle in between and relative to the diameter adjacent to the first shoulder and the second shoulder, respectively 63 and 64.

In FIG. 14 the female element 60F is shown as a tube having an inner diameter and terminated by a cutout resulting from a partial radial cut perpendicular to the female element 60F and a further axial and longitudinal cut through one wall of the tube. Thereby a free hanging tongue 66 is provided, which is plastically deformed from a circular shape so as to form a spiral, which is configured to perform as a radially curved leaf spring.

Since the connector 60 couples axially, the axial width of the tongue 66 is selected to match the length of the groove 62 for snap-in reception therein.

The inner diameter of the spiraling tongue 66 evidently varies but is configured to accommodate the outer diameter of the ogive 61 and ensure elastic deformation of the tongue 66 when the ogive 61 of the male element 60M is axially introduced therein. Once the male element 60M is received inside the female element 60F, the tongue 66 becomes firmly seated inside the groove 62 and snugly caught between the first shoulder 63 and the second shoulder 64.

A longitudinal axial passage through the connector 60 allows the insertion therethrough of a core wire 30.

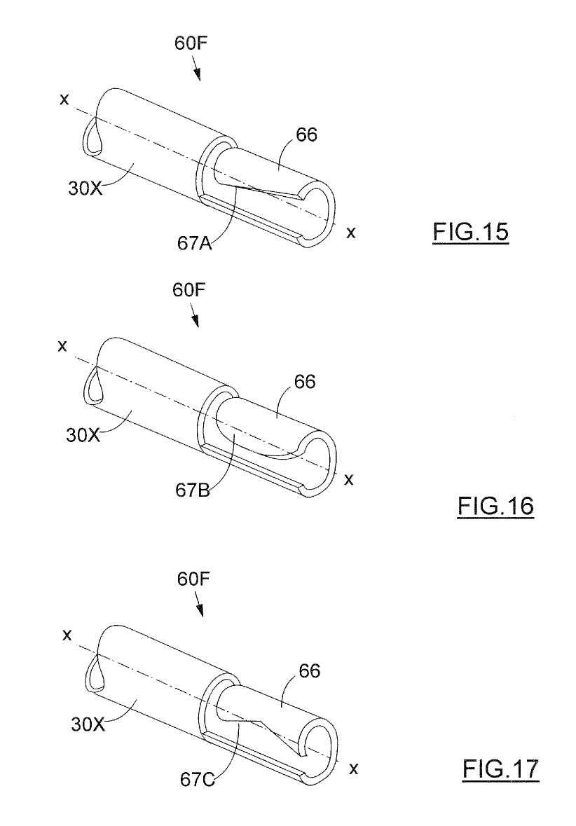

FIG. 15 shows a free hanging tongue 66 having a free edge 67A terminated at an angle relative to the axis X-X of the connector 60, whereas in FIG. 14 the free edge 67 is parallel to the axis X-X. Furthermore, in FIG. 16 the free edge 67A extends as a curve configured to match the varying radius of the bottom 65 of the groove 62.

Although not shown in the Figs., other configurations of the free hanging tongue 66 are acceptable, such as for example the concave or the cut-in free edge 67B shown in FIG. 16. Furthermore, the free hanging tongue 66 may be configured to form more than one leaf spring for engaging one grove 65 or more than one groove disposed on the male element 60M.

It is noted that the largest outer diameter of the ogive 61 must be smaller than the largest inner diameter achieved by deflection of the tongue 66 of the female element 60F.

FIG. 17 is shows a further embodiment of a free hanging tongue 66 with a free edge 67C in the shape of a fishtail.

Whatever the configuration of the male and female elements of the connector 60, respectively 60M and 60F, repetitive operation is ensured and the disconnection is fast and easy, by relative bending of one of the elements relative to the other.

FIG. 18 shows a rotation lockable connector 60R having a male element 60RM and a female element 60RF.

The male element 60RM is similar to the male element 60M but for the addition of a non-axisymmetric element that is coupled to the apex of the ogive 61. For example, as shown in FIG. 18, a flat plate 68 which may be axially coupled to the apex of the ogive 61.

The female element 6ORF presents the same free hanging curved tongue 66 radial leaf spring, not shown in FIG. 18, as with the female element 60F but further has a non-axisymmetric restriction 69, or female restriction 69, entered therein to accommodate the axial passage in translation of the flat plate 68 only when properly radially aligned therewith.

To couple the male element 6ORM to the female element 60RF, it is necessary to mutually align the female restriction 69 with the flat plate 68, and to introduce the male element 60RM into the female element 6ORF. This arrangement prevents mutual rotation between the wire 30 and the extension wire 30X. Disconnection requires to first axially-retrieve the flat plate 68 out of the female restriction 69.

A longitudinal axial passage through the length connector 60, thus also through the ogive 61, allows the insertion therethrough of a core wire 30.

In summary, a proximally disposed wire connector 60 or 60R, is provided for longitudinal axial quick-releasable connection and disconnection of an extension wire 30X, respectively to and from a guide wire 30 or from an equivalent guide 80 or 73 co-extensive thereto. The wire connector and the extension wire may be configured for smooth translation over the guide wire 30 or an equivalent guide, of an additional tool, for further in vivo treatment or operation. Furthermore, the wire connector may have a male element 60M or 6ORM which is configured to couple with a female element 60F or 6ORF, by help of at least one radial curved leaf spring, selected from a configuration such as 67A, 67B, or 67C.

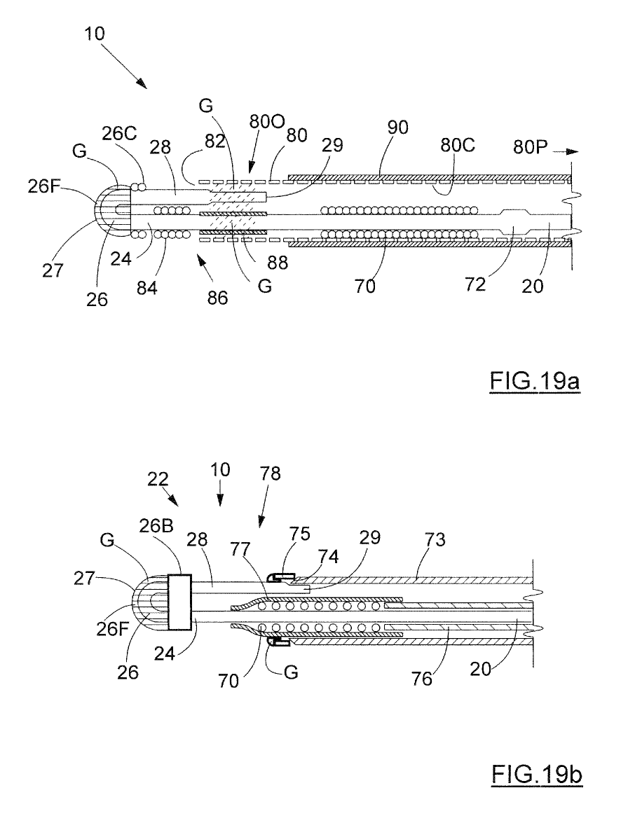

Yet another exemplary embodiment of the auger tool 10 is described in FIG. 19a, starting from the distal extremity.

FIG. 19a illustrates the distal portion of the auger tool 10 according to another embodiment. A fairing 26F covers the bend 26 to which abuts a wrapped coil 26C that is retained on the core-incoming portions 24 and on the core-returning portion 28. When glue, including UV glue is used with the fairing 26F, that same glue is preferably used to secure the wrapped coil 26C in place. Since the bend 26 is covered by a fairing 26F, the tool nose 27 is the most distal portion of the auger tool 10.

The core wire 20 is possibly made of a super-elastic material such as Nitinol, to impart superior properties thereto with regard to flexibility, resilience, and elongation. Other core distal portion 22 configurations, such as described with respect to the other embodiments described hereinabove are also applicable if desired.

In FIG. 19a, an elongated coil guide 80, the proximal end of which, not shown in FIG. 19a, is securely attached in retention to and in continuation of the guide wire 30, houses both the core-incoming portions 24 and the core-returning portion 28 that protrude distally out of the distal opening extremity 82 of the elongated coil guide 80. The coil guide 80 contributes to the flexibility and resiliency of the core distal portion 22, and is coiled with open pitch coils 80O at the distal elongated coil guide extremity 86, but mainly with closed pitch coils 80C proximally thereof, on the body of the coil guide 80. If desired, it is possible to take advantage of further open pitch coils 80O in between which glue may penetrate, at the proximal extremity 80P of the coil guide 80 to glue extension-connecting items thereto. The coil guide 80 may thus be regarded as being another configuration or an equivalent of the guide wire 30.

A stopper 84 may be appropriately disposed on the core-incoming portions 24 intermediate the distal opening extremity 82 and the wrapped coil 26C, and may act as a limiting stopper to constrain and limit the translation motion of the core distal portion 22. If desired, the stopper 84, as well as the wrapped coil 26C are implemented from radio opaque material used in common practice and well known in the art, such as platinum, whereby the disposition and the relative location of these components of the auger tool 10 are also presented to the operator when using and viewing an imaging device.

In the interior of the elongated coil guide 80 adjacent the distal elongated coil guide extremity 86, the incoming core 24 may be covered with a polymeric sleeve 88, such as for example a shrink tube. This ensures smooth translation of the incoming core 24 relative to the interior of the stainless steel elongated coil guide 80 to which the polymeric sleeve 88 is retained say by glue G.

The core distal extremity 29 is flattened to enhance retention to the interior of the distal elongated coil guide extremity 86 by any method known to the art, such as welding or gluing, including UV glue. When glued, the polymeric sleeve 88 disposed on the incoming core 24 forms a substrate for the application of the glue, G which may be supplied through the open pitch coils portion 80O disposed at the distal guide coil extremity 86. Furthermore, the polymeric sleeve 88 prevents any contact of spillovers of glue G with the incoming core wire 24.

If desired, the interior of the distal coil guide extremity 86 is potted with glue G inserted through the open pitch coils to enhance retention of the core distal extremity 29.

An exterior sleeve 90, say a polyester heat-shrink sleeve 90, may cover the closed pitch coils portion of the elongated coil guide 80 to prevent the extension of the coils when extension forces are applied thereto. Should such an elongation occur, then a portion of the restoring forces released by the main resilient element disposed in the auger actuator 500, not shown in FIG. 19a, would be wasted and by not being available, would prevent the return of the core distal portion 22 to the extended and straight state.

Another marker coil 70 is disposed, if desired, on the core wire 20 proximally to the polymeric sleeve 88 to enhance radio opaqueness and assist the operator OP. The marker coil 70 is coupled to the core wire 20 by means well known to the art, such as for example by gluing or by a local plastic deformation applied to the core 20 and shown symbolically as 72 on FIG. 19a.

FIG. 19b depicts another exemplary embodiment of the auger tool 10. For example, the core distal portion 22 may have a distal extremity with a fairing 26F, which covers the bend 26. A surrounding band 26B may abut the fairing 26F and be retained on the distal portion of both the core-incoming portions 24 and the core-returning portion 28. When glue G. including UV glue is used for forming the fairing 26F, that same glue is preferably used to fixedly attach the surrounding band 26B in place.

If desired, the surrounding band 26B may be configured as an opaque marker, or platinum marker, and the same is true for the fairing 26F. The configuration of the core distal portion 22 may be selected as desired, for example as described with respect to the FIGS. 9 to 13.

As already described hereinabove, the core wire 20 is possibly made of a super-elastic material such as Nitinol, to impart superior properties thereto with regard to flexibility, resilience, and elongation characteristics.

The core distal portion 22 protrudes out a distal portion of a flexible tube 73, or a flexible coil 73, which is distally terminated by a taper 74. A marker sleeve 75, operative as an opaque marker may be disposed in longitudinal continuation to the flexible tube 73 in abutment to the taper 74. The core distal extremity 29 is coupled to the distal extremity of the flexible tube 73 as described hereinabove.

In distanced separation away from the surrounding band 26B, the distal extremity of the core-incoming portion 24 is covered by a short portion of coil 70, possibly a marker coil 70, having a distal portion, which protrudes out of the distal extremity of the flexible tube 73, and a proximal portion that is disposed in the interior of the flexible tube 73.

In proximal continuation of the marker coil 70 and in abutment therewith, a continuation tube 76, possibly made of Nitinol or of stainless steel is coupled thereto by a shrunken polymeric sleeve 77 overlapping the length of the coil 70 and a distal portion of the continuation tube 76. The continuation tube 76 prevents proximal displacement of the marker coil 70. The distal portion of the polymeric sleeve 77 shrinks onto the core-incoming portion, and the proximal portion of the polymeric sleeve 77 shrinks onto the continuation tube 76, whereby the marker coil to is sealed from the surroundings.

The distal portion 78 of the marker sleeve 75 may be potted or glued to the core-returning portion 28 and to the polymeric sleeve 77.

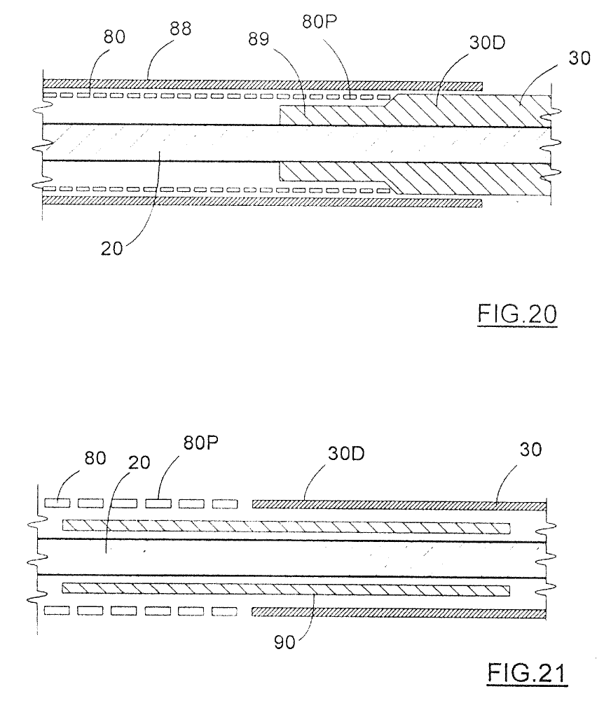

FIG. 20 depicts details of the coupling connection between the proximal portion 80P of the elongated coil guide 80 with the distal portion 30D of the guide wire 30. The distal portion 30D has an appropriately reduced diameter portion 89 for insertion into the interior of the proximal portion 80P and for firm retention in translation and rotation by any of the methods known to the art. For example, by welding or gluing, and by help of the polymeric sleeve 90 to also cover the distal portion 30D.

FIG. 21 illustrates an alternative method for coupling the proximal portion 80P of the elongated coil guide 80 to the distal portion 30D of the guide wire 30 by use of an intermediate sleeve 90 that is inserted in a portion of the interior of both the proximal portion 80P and the distal portion 30D as a substrate for the glue G retaining both of them. If desired, the intermediate sleeve 90 is made from super-elastic metal such as Nitinol, which will help to maintain coaxial alignment between both the proximal portion 80P and the distal portion 30D. The core wire 30 is free to translate in the interior of the intermediate sleeve 90. It is noted that the rigidity of the intermediate sleeve 90 may be modified according to needs by application of appropriate skiving or laser cutting.

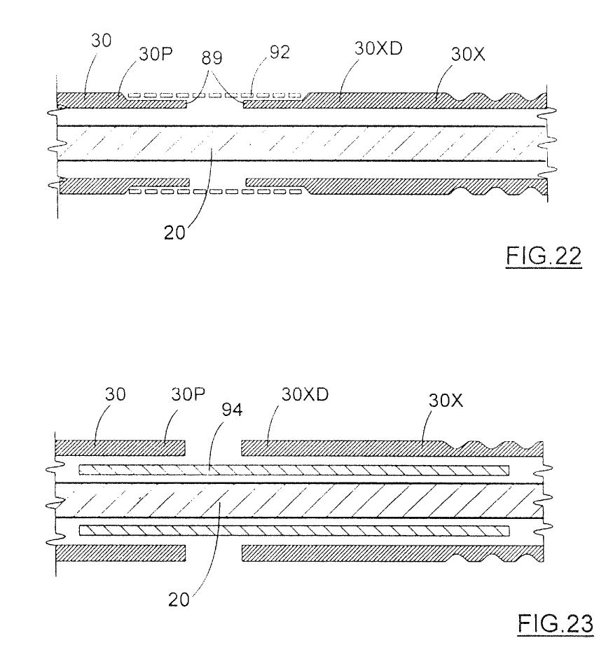

FIG. 22 depicts details of the fixed-retention coupling connection between the proximal end 30P of the guide wire 30 and the distal end 30XD of an extension wire 30X whereover a cover coil 92 is engaged. The proximal end 30P and the distal end 30XD have a reduced diameter portion 89 appropriately configured to accommodate insertion into the cover coil 92 engaged over the reduced diameter 89 and fixedly coupled thereto by means known to the art, for example, by welding, or by gluing. The cover coil 92 ensures a smooth external surface at the transition region over the guide wire 30 and the extension wire 30X, and facilitates passage thereover of catheters and other equipment. If it is desired to prevent relative translation and/or rotation between the core wire 20 and the extension wire 30X, it is possible to crimp the extension wire 30 into plastic deformation onto the core wire 20, or use any other process to that end.

FIG. 23 illustrates an alternative method for fixedly coupling the proximal end 30P of the guide wire 30 to the distal end 30XD of an extension wire 30X by use of an insert coil 94 that is inserted into a portion of the interior of both the proximal portion 30P of the guide wire 30 and the distal portion 30XD as a substrate for the glue G retaining both of them. If it is desired to prevent relative translation and/or rotation between the core wire 20 and the guide wire 30, it is possible to crimp the guide wire 30 into plastic deformation onto the core wire 20, or use any other process therefore. Although shown in FIG. 23 as such, there is no gap between the proximal end 30P and the distal end 30XD.

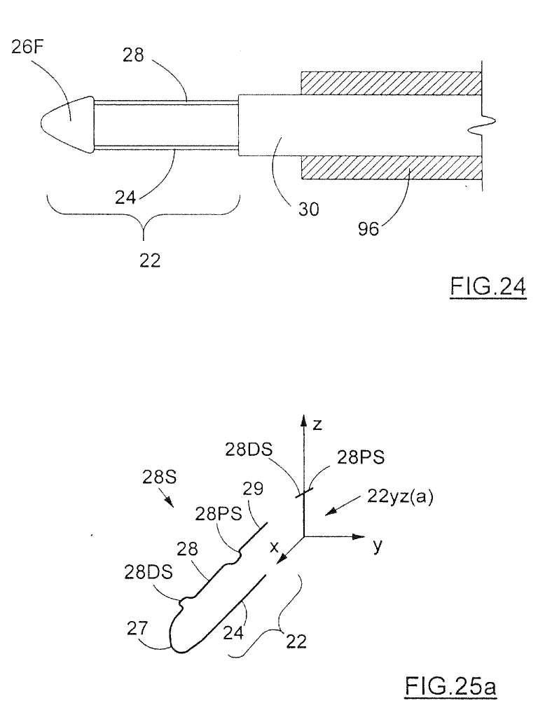

FIG. 24 shows a detail of an example of and additional tool possibly guided over the guide wire 30. A support tube 96 is appropriately configured and disposed over the wire 30 to provide additional support. In other words, at least one additional treatment tool may be translated over the auger tool, for further in vivo operation.

The deflection of the various embodiments of the auger tool 10 described hereinabove are not limited and restricted to the two-dimensional plane defined by the core-incoming portion 24, the bend 26, and the core-returning portion 28. Rather, if desired, the auger tool 10 may be configured to deflect in a three-dimensional mode. Three-dimensional deflection is initiated by providing for at least one plastic deformation such as forming a saliency 28S disposed on the core-returning portion 28.

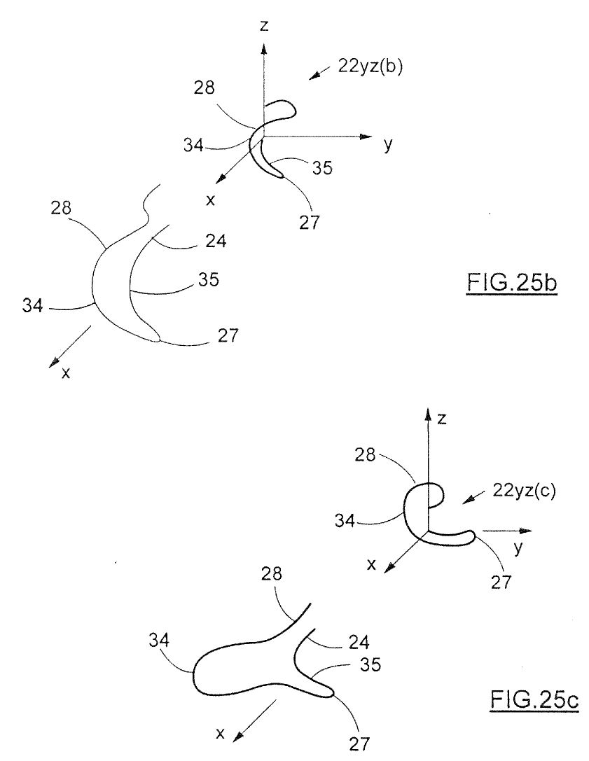

FIG. 25a illustrates an exemplary embodiment showing only the core wire 20 of the core distal portion 22 up to the core wire extremity 29 for the sake of clarity. Reference is made to a set of right-hand Cartesian coordinates. In the core distal portion 22, the core-incoming portion 24 remains straight but the core-returning portion 28 is configured with two tiny plastic deformations, which are formed thereon to initiate a three-dimensional deflection when operated to deflect. The longitudinally disposed core-returning portion 28 remains substantially straight but for a first proximal saliency 28PS separated apart by a distance from a second distal saliency 28DS. Both saliencies 28PS and 28DS are disposed distally away from the core wire extremity indicated as numeral 29.

As shown in FIG. 25a, both saliencies, respectively 28PS and 28DS, may be formed such as to be disposed in a same plane, but may also be disposed in different planes, where each saliency 28S projects in oblique to an opposite side of the straight plane defined by the core-incoming portion 24 and the core-returning portion 28. For example, when the core distal portion 22 is disposed along the x-axis in the vertical plane x-z according to the set of right-hand coordinates shown in FIG. 25a, then the two saliencies, respectively 28PS and 28DS, may be disposed in an oblique plane, say in oblique to the plane x-z. Such an exemplary disposition is indicated by a projection onto the y-z plane, as curve 22yz(a) shown in FIG. 25a. It is emphasized that the FIG. 25a, as well as all the other Figs., is not to scale and that the size of both saliencies 28S is greatly exaggerated for the sake of clarity.

FIG. 25b depicts one exemplary stage of deflection of the initially longitudinally disposed core distal portion 22, which deflects asymmetrically in three dimensions in relation to the set of right-hand Cartesian coordinates. When deflected out of the released state, say out of the x-z plane, the core distal portion 22 deflects asymmetrically, with the core-returning portion obtaining a bell-shaped contour that crosses both the x-z and the x-y planes. In this state of deflection, the extrados depression ED, and the tool bend 26 or tool nose 27, are disposed on either side of the x-z plane. The extrados depression ED is thus shown to reside in the quadrant (x, -y, z) while the tool bend 26 or tool nose 27 penetrate in the quadrant (x, y, -z). A projection on the y-z plane, shown as 22yz(b), attempts to visualize the complex asymmetrical deflection of the core distal portion 22.

The three-dimensional deflection shown in FIG. 25b is in contrast with the embodiments described hereinabove, where the deflection of the core distal portion 22 remains in the same plane when deflected and when released.

When further deflected, as shown in FIG. 25c, the longitudinally oriented core distal portion 22 may reach a lateral dilation disposed mainly in the x-y plane, thus a dilation perpendicular to the plane defined by the core wire 20 of the longitudinal core distal portion 22. This means that both the extrados depression ED and the nose depression ND reside substantially in the x-y plane. In practice for example, with a core wire 20 having a diameter of some 0.1 mm, the height of the core distal portion 22 when in the released state is about 0.3 mm. However, when deflected, this last embodiment allows an asymmetric lateral deflection of some 1.4 mm.

The core-incoming portion and the core-returning portion may define a released-plane when the core distal portion is disposed in the released state, and when at least one permanent plastic deformation is made to the core incoming portion to induce an out-of-released-plane deflection shape is achieved when the auger tool is operated to deflect. Preferably, two permanent plastic deformations are made to induce an out-of-released-plane deflection shape the core-incoming portion. Thereby the auger tool will deflect into a three-dimensional out-of-released-plane deflection when the auger tool is operated to the deflected state.

Thus, when the auger tool is appropriately deflected, the extrados and the tool nose are disposed in a deflected plane, which deflected plane is substantially perpendicular to the released-plane. Possibly, each one of the extrados and of the tool nose are disposed in a separate and different deflected plane which is inclined at an angle relative to the released-plane. Alternatively, when the auger tool is appropriately deflected, the extrados will be disposed in a first plane and the tool nose will be disposed in a second plane, with each one of the first plane and of the second plane defining a separate different deflected plane, which is inclined at an angle relative to the released-plane.

### INDUSTRIAL APPLICABILITY

The occlusion auger described hereinabove is configured for manufacture in industry, and in particular in the medical instruments producing industry.

It will be appreciated by persons skilled in the art, that the present invention is not limited to what has been particularly shown and described hereinabove. For example, the auger tool may have other configurations, as long as atraumatic distal deflective rolling motion is provided. Furthermore, the auger tool 10, which has at least one flexible and deflectable portion, may have more such portions, such as for example more than one core wire 20, more than one core-incoming portions 24, and more than one core-returning portions 28. Rather, the scope of the present invention is defined by the appended claims and includes both combinations and sub-combinations, for example of the many embodiments of the core distal portion, of the various features described hereinabove as well as variations and modifications thereof which would occur to persons skilled in the art upon reading the foregoing description.

### LIST OF ITEMS

10 distal auger tool
20 core wire
22 core distal portion
22P core proximal portion
24 core-incoming portion
26 bend

26B surrounding band

26C wrapped coil

26F fairing cover

26S stiffened bend

26W weld
27 tool nose

27W weld
28 core-returning portion

28S saliency

28PS proximal saliency

28DS distal saliency
29 core distal extremity
30 guide wire

30D distal portion of the guide wire 30

30I interior of the wire 30

30P proximal end of the guide wire 30

30X extension wire 30X

30XD distal end of an extension wire 30X
32 distal wire opening
32 interior of the wire 30
34 extrados
35 intrados
60 wire connector

60M male element

60F female element

60R rotation lockable connector
60RM male element
60RF female element
61 ogive
62 groove
63 first shoulder
64 second shoulder
65 bottom of the groove 62
66 free hanging tongue
67 free edge

67A

67B

67C
68 flat plate
69 non-axisymmetric restriction
70 marker coil
71
72 local plastic deformation
73 flexible tube or a flexible coil
74 taper
75 marker sleeve
76 continuation tube
77 polymeric sleeve
78 distal portion of the marker sleeve 75
80 coil guide
80C closed pitch coils 80C
80O open pitch coils
80P proximal extremity of the coil guide 80
82 distal opening extremity
84 stopper
86 coil guide extremity
88 polymeric sleeve 88
89 reduced diameter portion
90 exterior sleeve
92 cover coil
94 insert coil
96 support tube
300 blood vessel
310 vessel walls
320 occlusion
340 furrow
500 auger actuator
502 main resilient actuator element
G glue
ND nose depression
ED extrados depression

1. A method for implementing an occlusion auger operated proximally by an auger actuator and adapted for distally traversing an occlusion, the occlusion auger comprising:
a distal auger tool including a guide wire and a core wire introduced through a first proximal wire opening entered in the guide wire and exiting out of a second distal wire opening as a core distal portion,
the method being characterized by comprising the steps of:
configuring the core wire exiting out of an interior of the guide wire as a distal core-incoming portion which is terminated by an arc to form a bend,
configuring the portion of the core wire extending from the bend and returning back into the distal wire opening as a distal core-returning portion which is terminated by a core distal extremity,
firmly securing the core distal extremity in the interior of the guide wire at the distal guide wire opening, and
configuring the resilient core wire as a unitary piece of material having the distal extremity core portion operative as an auger tool and a proximal extremity core portion operative as a force and motion transmitting element operated directly by the auger actuator, to minimize auger tool dimensions and increase operational reliability.

2. The method according to claim 1, wherein:
the core distal portion is urged into an arcuate state by the auger actuator, for the core-returning portion to deform in a longitudinal continuous extrados and the core-incoming portion to deform into a longitudinal continuous intrados

3. The method according to claim 2, wherein:
the core distal portion has a tool nose which is the most distal portion of the auger tool, and
in the arcuate state of the core distal portion, the tool nose and the extrados are disposed in opposite to each other in spaced-apart relationship.

4. The method according to claim 2, wherein:
first, the core-returning portion is in direct operative association with the guide wire, to flex the core returning portion to the arcuate state by translating the guide wire distally relative to the auger actuator, for the extrados to dilate in asymmetric radial outward direction, and
second, the core-returning portion is released to the released state by release of the guide wire by the auger actuator.

5. The method according to claim 1, wherein:
the core distal portion is disposed in coextensive alignment with the wire at rest in a released extended and straightened-out state, and
the core-returning portion is flexed into an arcuate state extending radially away from the wire by the auger actuator which induces buckling of the core-returning portion and curving of the core incoming portion by decrease of the distance separating the core distal extremity from the bend.

6. The method according to claim 1, wherein:
the core-retuning portion is configured to have a first position as a normally extended state, a second position as a flexed state, and an intermediate position wherein the auger tool is dispose in a navigation state.

7. The method according to claim 6, wherein:
the normally extended state, the flexed state and the navigation state of the core distal portion are adapted to initiate a crack propagation mechanism.

8. The method according to claim 6, wherein:
in the navigation mode:
the core-returning portion is in partial deflection intermediate the fully extended and straightened state and the fully arcuate state,
the core distal portion is deflected radially away relative to a longitudinal axis relative to the distal portion of the guide wire to form therewith an angle α, and
rotation of the guide wire rotates the core wire and the core-incoming portion, to become a directrix describing a mantle of a cone permitting to orient the bend in any desired direction to properly aligning the auger tool into sideways branching.

9. The method according to claim 1, wherein:
flexure of the core-returning portion is delimited between the bend and the core distal extremity, and
coupling of the core distal extremity to the guide wire serves as a incontinuous point connection where flexing starts.

10. The method according to claim 1, wherein:
flexure of the core-returning portion is delimited between the bend and the core distal extremity, and
the bend is stiffened for the core-returning portion to be less rigid relative to the bend and to deflect adjacent thereto.

11. The method according to claim 1, wherein:
the incoming portion and the returning portion are coupled by a connection selected alone and in combination at least from the group consisting of a bend, a surrounding band, a wrapped coil, a fairing cover, and a weld, to relieve stress at the bend.

12. The method according to claim 11, wherein:
the selected connection is implemented from or includes a radio opaque material.

13. The method according to claim 1, wherein:
an elongated coil guide is securely attached to and in continuation of the guide wire to house both the core-incoming portions and the core-retuning portion protruding distally out of a distal opening extremity of the elongated coil guide, and
the coil guide is coiled with open pitch coils at a distal elongated coil guide extremity and with closed pitch coils proximally thereof, on the body of the coil guide,
whereby the coil guide contributes to the flexibility and resiliency of the core distal portion.

14. The method according to claim 1, wherein:
at least one plastic deformation is disposed on the core-returning portion,
whereby the auger tool is configured to deflect in three-dimensions.

15. The method according to claim 1, wherein:
at least two plastic deformations are formed on the core-returning portion, both deformations being formed in at least one plane,
whereby the auger tool is configured to deflect in three-dimensions.

16. An occlusion auger system operated proximally by an auger actuator and adapted for distally traversing an occlusion, the occlusion auger system comprising:
a distal auger tool including a guide wire and a core wire introduced through a first proximal wire opening entered in the guide wire and exiting out of a second distal wire opening as a core distal portion,
the system being characterized by comprising:
a distal core-incoming portion configured out of the core wire exiting out of an interior of the guide wire terminated by an arc to form a bend,
the portion of the core wire extending from the bend and returning back into the distal wire opening being configured as a distal core-returning portion which is terminated by a core distal extremity,
the core distal extremity being firmly secured in the interior of the guide wire at the distal guide wire opening, and
the resilient core wire being configured as a unitary piece of material having the distal extremity core portion operative as an auger tool and a proximal extremity core portion operative as a force and motion transmitting element operated directly by the auger actuator, to minimize auger tool dimensions and increase operational reliability.

17. The system according to claim 16, wherein:
the core distal portion is urged into an arcuate state by the auger actuator, for the core-returning portion to deform in a longitudinal continuous extrados and the core-incoming portion to deform into a longitudinal continuous intrados.

18. The method according to claim 17, wherein:
the core distal portion has a tool nose which is the most distal portion of the auger tool, and
in the arcuate state of the core distal portion, the tool nose and the extrados are disposed in opposite to each other in spaced-apart relationship.

19. The system according to claim 17, wherein:
first, the core-returning portion is in direct operative association with the guide wire, to flex the core returning portion to the arcuate state by translating the guide wire distally relative to the auger actuator, for the extrados to dilate in asymmetric radial outward direction, and
second, the core-returning portion is released to the released state by release of the guide wire by the auger actuator.

20. The system according to claim 16, wherein:
the core distal portion is disposed in coextensive alignment with the wire at rest in a released extended and straightened-out state, and
the core-returning portion is flexed into an arcuate state extending radially away from the wire by the auger actuator which induces buckling of the core-returning portion and curving of the core incoming portion by decrease of the distance separating the core distal extremity from the bend.

21. The system according to claim 16, wherein:
the core-returning portion is configured to have a first position as a normally extended state, a second position as a flexed state, and an intermediate position wherein the auger tool is dispose in a navigation state.

22. The system according to claim 21, wherein:
the normally extended state, the flexed state and the navigation state of the core distal portion are adapted to initiate a crack propagation mechanism.

23. The system according to claim 21, wherein:
in the navigation mode:
the core-returning portion is in partial deflection intermediate the fully extended and straightened state and the fully arcuate state,
the core distal portion is deflected radially away relative to a longitudinal axis relative to the distal portion of the guide wire to form therewith an angle α, and
rotation of the guide wire rotates the core wire and the core-incoming portion, to become a directrix describing a mantle of a cone permitting to orient the bend in any desired direction to properly aligning the auger tool into sideways branching.

24. The system according to claim 16, wherein:
flexure of the core-returning portion is delimited between the bend and the core distal extremity, and
coupling of the core distal extremity to the guide wire serves as a incontinuous point connection where flexing starts.

25. The system according to claim 16, wherein:
flexure of the core-returning portion is delimited between the bend and the core distal extremity, and
the bend is stiffened for the core-returning portion to be less rigid relative to the bend and to deflect adjacent thereto.

26. The system according to claim 16, wherein:
the incoming portion and the returning portion are coupled by a connection selected alone and in combination at least from the group consisting of a bend, a surrounding band, a wrapped coil, a fairing cover, and a weld, to relieve stress at the bend.

27. The system according to claim 26, wherein:
the selected connection is implemented from or includes a radio opaque material.

28. The system according to claim 16, wherein:
an elongated coil guide is securely attached to and in continuation of the guide wire to house both the core-incoming portions and the core-returning portion protruding distally out of a distal opening extremity of the elongated coil guide, and
the coil guide is coiled with open pitch coils at a distal elongated coil guide extremity and with closed pitch coils proximally thereof, on the body of the coil guide,
whereby the coil guide contributes to the flexibility and resiliency of the core distal portion.

29. The system according to claim 16, wherein:
at least one plastic deformation is disposed on the core-returning portion,
whereby the auger tool is configured to deflect in three-dimensions.

30. The system according to claim 16, wherein:
at least two plastic deformations are formed on the core-returning portion, both deformations being formed in at least one plane,
whereby the auger tool is configured to deflect in three-dimensions.
